SEQUENCE LISTING (2 sequences)

SEQ ID NO: 1 - DNA, 345 bp, synthetic construct
  misc_feature 1..345: note = Synthetic: XCL1-expressing nucleic acid
  source 1..345: mol_type = other DNA
atgaggctgctgatcctggccctgctgggcatctgcagcctgaccgcctacatcgtggagggcgtgggcagcgaggtgagcgacaagaggacctgcgtgagcctgaccacccagaggctgcccgtgagcaggatcaagacctacaccatcaccgagggcagcctgagggccgtgatcttcatcaccaagaggggcctgaaggtgtgcgccgacccccaggccacctgggtgagggacgtggtgaggagcatggacaggaagagcaacaccaggaacaacatgatccagaccaagcccaccggcacccagcagagcaccaacaccgccgtgaccctgaccggctag

SEQ ID NO: 2 - protein, 114 aa, synthetic construct
  REGION 1..114: note = Synthetic: polypeptide encoded by XCL1-expressing nucleic acid
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

(19) United States
(12) Patent Application Publication
(10) Pub. No.: US 2026/0337281 A1
(43) Pub. Date: Oct. 29, 2026

(54) COMPOSITIONS AND METHODS FOR MODULATING T CELL FUNCTION

(71) Applicant: The Regents of the University of California, Oakland, CA (US)

(72) Inventors: Amir FERRY, La Jolla, CA (US); Ananda GOLDRATH, San Diego, CA (US)

(21) Appl. No.: 19/115,761

(22) PCT Filed: Sep. 26, 2023
(86) PCT No.: PCT/US2023/033744

§ 371 Date: Mar. 26, 2025

Related U.S. Application Data
(60) Provisional application No. 63/410,021, filed on Sep. 26, 2022.

(51) Int. Cl. C07K 16/28; A61K 35/17; A61K 40/11; A61K 40/31; A61K 40/42; A61P 35/00; C07K 14/52; C12N 5/0783; C12N 15/86

(52) U.S. Cl. CPC C07K 16/2866; A61K 35/17; A61K 40/11; A61K 40/31; A61K 40/4219; A61K 40/4224; A61P 35/00; C07K 14/521; C07K 16/2809; C12N 5/0636; C12N 15/86; A61K 2239/13; A61K 2239/29; C07K 2317/31; C12N 2510/00; C12N 2750/14143

(57) ABSTRACT

In alternative embodiments, provided are compositions, including compositions, products of manufacture and kits, and methods, for manipulating, and in particular, augmenting or increasing, T cell activity in vitro and in vivo, by for example increasing XCL1 gene expression in T cells in vivo, or by increasing the number of XCL1 expressing T cells in vivo, or by administering to an individual in need thereof. T cells (such as CAR T cells) genetically manipulated to have increased XCL1 polypeptide expression.

### RELATED APPLICATIONS

This Patent Convention Treaty (PCT) International Application claims the benefit of priority under 35 U.S.C. § 119(e) of U.S. Provisional Application Serial No. (USSN) 63/410,021, filed Sep. 26, 2022. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under AI132122 and AI072117 awarded by the National Institutes of Health (NIH). The government has certain rights in the invention.

### TECHNICAL FIELD

This invention generally relates to infectious diseases and immunoassays. In alternative embodiments, provided are compositions, including compositions, products of manufacture and kits, and methods, for manipulating, and in particular, augmenting or increasing, T cell activity in vitro and in vivo, by for example increasing XCL1 gene expression in T cells in vivo, or by increasing the number of XCL1 expressing T cells in vivo, or by administering to an individual in need thereof. T cells (such as chimeric antigen receptor (CAR)-T cell) genetically manipulated to have increased XCL1 polypeptide expression.

### BACKGROUND

Chemokine ligand XCL1, also known as lymphotactin, is a cytokine belonging to the C chemokine family. In normal tissues, XCL1 is found in high levels in spleen, thymus, small intestine and peripheral blood leukocytes, and at lower levels in lung, prostate gland and ovary. Secretion of XCL1 is responsible for the increase of intracellular calcium in peripheral blood lymphocytes. Cellular sources for XCL1 include activated thymic and peripheral blood CD8+ T cells. NK cells also secrete XCL1 along with other chemokines early in infections.

The pair of XCL1 and its receptor XCR1 are known to be involved in cross-presentation, antigen uptake, and induction of innate as well as adaptive cytotoxic immunity. XCR1, the receptor for XCL1, is exclusively expressed in conventional dendritic cells. XCL1 is secreted by NK cells and by antigen-specific CD8+ T-cells, along with other chemokines including IFN-gamma. This process likely facilitates the cross-presentation of antigens by the dendritic cells.

XCL1 is also known to increate T cells in joints that are effected with rheumatoid arthritis (RA). They are also expressed on RA synovial lymphocytes.

XCL1 has been found to be expressed on tumor cells in mature cystic teratoma of the ovary (MCT)− squamous cell carcinomas (SCC). XCL1 expression has been shown to be significantly associated with the number of tumor-infiltrating CD8-positive T cells and PD-L1 expression on tumor cells. XCL1 produced by tumor cells may induce PD1/PD-L1 interaction and dysfunction of CD8-positive T cells in tumor microenvironment. XCL1 expression may be a novel biomarker for malignant transformation of MCT into SCC and a biomarker candidate for therapeutic response to an anti-PD1/PD-L1 therapy.

### SUMMARY

In alternative embodiments, provided are methods for:
(i) treating, ameliorating, slowing the progression of, decreasing the severity of symptoms of, or preventing a tumor or a cancer,
(ii) stimulating T cell generation or activity, optionally stimulating cancer-targeting T cell generation or activity, or
(iii) recruiting XCR1⁺ conventional type 1 DC (dendritic cell) (cDC1) cells to improve an immune response, wherein optionally the immune response is an anti-cancer immune response,
comprising administering to an individual in need thereof:
(a) a nucleic acid encoding a XCL1, or lymphotactin, polypeptide, wherein optionally the XCL1-expressing nucleic acid is an XCL1 gene or XCL1 mRNA sequence, or the XCL1-expressing nucleic acid is an RNA or a DNA molecule, or a synthetic nucleic acid,
(b) a T cell genetically manipulated or engineered to have increased XCL1 polypeptide expression, wherein optionally the T cell is a chimeric antigen receptor (CAR)-T cell, and optionally the CAR-T cell targets cancer cells, or
(c) a bispecific antibody or a bispecific antigen binding polypeptide, wherein the bispecific antibody or the bispecific antigen binding polypeptide can simultaneously specifically bind: (i) an XCR1 (receptor for XCL1) expressed on an outer surface of a T cell, and (ii) a T cell receptor (TCR) or CD3 protein complex on the surface of the T cell,
wherein optionally the XCL1-expressing nucleic acid comprises the nucleic acid sequence (SEQ ID NO:1):

ATGAGGCTGCTGATCCTGGCCCTGCTGGGCATCTGCAGCCTGACCGCCT
ACATCGTGGAGGGCGTGGGCAGCGAGGTGAGCGACAAGAGGACCTGCGT
GAGCCTGACCACCCAGAGGCTGCCCGTGAGCAGGATCAAGACCTACACC
ATCACCGAGGGCAGCCTGAGGGCCGTGATCTTCATCACCAAGAGGGGCC
TGAAGGTGTGCGCCGACCCCCAGGCCACCTGGGTGAGGGACGTGGTGAG
GAGCATGGACAGGAAGAGCAACACCAGGAACAACATGATCCAGACCAAG
CCCACCGGCACCCAGCAGAGCACCAACACCGCCGTGACCCTGACCGGCT
AG

or a nucleic acid having between about 90% to 99% sequence identity to SEQ ID NO:1 and encoding a polypeptide having XCL1 chemokine activity,
and optionally the XCL1-expressing nucleic acid comprises a nucleic acid sequence that encodes the polypeptide:

(SEQ ID NO: 2)
MRLLILALLGICSLTAYIVEGVGSEVSDKRTCVSLTTQRLPVSRIKTYT
ITEGSLRAVIFITKRGLKVCADPQATWVRDVVRSMDRKSNTRNNMIQTK
PTGTQQSTNTAVTLTG

or a polypeptide having between about 90% to 99% sequence identity to SEQ ID NO:2, or a polypeptide having about 92%, 95% or 97% sequence identity to SEQ ID NO:2 and having XCL1 chemokine activity,
wherein optionally the sequence identities (SEQ IDs) are determined by analysis with a sequence comparison algorithm or by a visual inspection, and optionally the sequence comparison algorithm is a BLAST version 2.2.2 algorithm where a filtering setting is set to blastall-p blastp-d “nr pataa”-F F, and all other options are set to default.

In alternative embodiments of methods as provided herein:
the XCL1-encoding nucleic acid, the T cell genetically manipulated or engineered to have increased XCL1 polypeptide expression, or the bispecific antibody or bispecific antigen binding polypeptide, is contained in a liposome, a liposomal vesicle, an expression construct, a plasmid, an expression vehicle, a virus or a vector, and the expression construct, plasmid, expression vehicle, virus or vector is delivered or administered to the individual in need thereof,
wherein optionally the expression vehicle or vector is selected from the group consisting of a herpes simplex virus, a human immunodeficiency virus (HIV), a synthetic vector, an adeno-associated virus (AAV), a lentivirus, an adenovirus and a plasmid,
and optionally the AAV is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant;
the XCL1-expressing nucleic acid, the expression construct, plasmid, expression vehicle, virus or vector, or the bispecific antibody or bispecific antigen binding polypeptide, or the genetically manipulated or engineered T cell, is formulated:
(a) in a liquid, a gel, a hydrogel, a vesicle, a liposome, a liposomal vesicle, a nanoparticle, a nanolipid particle, a powder or an aqueous or a saline formulation, or for administration in vitro or in vivo;
(b) for enteral or parenteral administration;
(c) in or as a liposome, a liposomal vesicle, a nanoparticle, or a nanoliposome;
(d) in or as a tablet, a pill, a capsule, a gel, a hydrogel, a geltab, a liquid, a powder, an emulsion, a lotion, an aerosol, a spray, a lozenge, an aqueous or a sterile or an injectable solution, an eye drop, or an implant; or
(e) for intravenous injection, subcutaneous injection, intrathecal injection, intramuscular injection, inhalation, or intravitreal injection; and/or
the nucleic acid encoding the XCL1 polypeptide, or the XCL1 gene or XCL1 mRNA sequence, or the XCL1-expressing RNA or DNA nucleic acid, or the expression construct, plasmid, expression vehicle, virus or vector, or the genetically manipulated or engineered T cell, or the bispecific antibody or bispecific antigen binding polypeptide, is formulated:
in a lipid formulation or a liposome or equivalents, as a nanoparticle, or a nanoliposome, and optionally the lipid formulation or equivalent is injected, optionally is injected intramuscularly (IM), or into or approximate to a solid tumor,
in a lipid formulation as described in U.S. patent application no. US 20210046173 A1, and/or
in a lipid formulation comprising or formulated as a liposome, in a liposomal vesicle, or a lipid nanoparticle (LNP), or nanoliposome, that comprises: non-cationic lipids comprise a mixture of cholesterol and 1,2-distearoyl-sn-glycero-3-phosphocholine (DSPC), DSPC alone, 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG), or a polyethylene glycol (PEG)-lipid, or PEG-modified lipid, or an ionizable cationic lipid.

In alternative embodiments provided are uses of a XCL1-expressing nucleic acid, or an expression construct, plasmid, expression vehicle, virus or vector as provided herein, or as having contained therein an XCL1-expressing nucleic acid, or a T cell genetically manipulated or engineered to express or overexpress an XCL1 polypeptide, or a liposome, a nanoparticle, or a nanoliposome having contained therein an XCL1-expressing nucleic acid, or the bispecific antibody or bispecific antigen binding polypeptide, for:
(a) treating, ameliorating, slowing the progression of, decreasing the severity of symptoms of, or preventing a tumor or a cancer,
(b) stimulating T cell generation or activity, or
(c) recruiting XCR1⁺ conventional type 1 DC (dendritic cell) (cDC1) cells to improve an immune response, wherein optionally the immune response is an anti-cancer immune response.

In alternative embodiments, provided are a XCL1-expressing nucleic acid, or an expression construct, plasmid, expression vehicle, virus or vector as provided herein, or as having contained therein an XCL1-expressing nucleic acid, or a T cell genetically manipulated or engineered to express or overexpress an XCL1 polypeptide, or a liposome, a nanoparticle, or a nanoliposome having contained therein an XCL1-expressing nucleic acid, for use in:
(a) treating, ameliorating, slowing the progression of, decreasing the severity of symptoms of, or preventing a tumor or a cancer,
(b) stimulating T cell generation or activity, or
(c) recruiting XCR1⁺ conventional type 1 DC (dendritic cell) (cDC1) cells to improve an immune response, wherein optionally the immune response is an anti-cancer immune response.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 schematically illustrates the relationship of T cells with environmental and physiologic stimuli, including transcriptional programs, environmental cues, cell to cell interactions, epigenetic remodeling and cell metabolism.

FIG. 2 schematically illustrates the cancer-immunity cycle and cell markers for identifying them, including: (1) release of cancer cells antigens, (2) cancer antigen presentation, (3) priming and activation of the immune system, (4) trafficking of T cells to tumors, (5) infiltration of T cells into tumors, (6) recognition of cancer cells by T cells; and (7) killing of cancer cells by T cells.

FIG. 3A-B graphically illustrate identifying the biological determinants of robust anti-tumor immune response:

FIG. 3A graphically illustrates progression-free survival; and

FIG. 3B graphically illustrates (upper image) patients achieving Minimal Residual Disease (MRD)-negative remission as a function of leukemia-free survival (%); and (lower image) all patients enrolled as a function of overall survival (%).

FIG. 4 illustrates a table showing the most frequently expressed genes in tumor Tissue Resident Memory CD8+ T cells (T_{RM}).

FIG. 5-F schematically illustrate the relationship of RNA-sequencing data to identify conserved genes in T cells: including the relationship between core Tissue Resident Memory CD8+ T cells (T_{RM}), TGFβ related, and progenitor exhausted T cells, with the marker Chemokine (C motif) ligand (XCL1) common to all:

FIG. 5A illustrates is a Venn diagram of differentially expressed genes derived from bulk RNA-seq data;

FIG. 5B graphically illustrates scRNA-seq displaying XCL1 expression in P14 CD8⁺ T_{RM} during D35 post LCMV-Arm infection;

FIG. 5C graphically illustrates gene expression analysis of XCL1 within Id3/Blimp1-expressing P14 populations in small intestine during LCMV-Arm (left data image) and tumor infiltrating lymphocytes during B16-GP₃₃₋₄₁ melanoma (right) data image;

FIG. 5D graphically illustrates expression of XCL1 and XCL2 in human CD8+ T cells sorted from jejunum and healthy donor blood;

FIG. 5E illustrates ATAC-seq data of XCL1 locus in CD8+T_{RM} population; and

FIG. 5F illustrates a confocal microscopy image of murine small intestine following P14 adoptive transfer and LCMV-Arm (D8 and D30 post infection), scale bar shown.

FIG. 6 schematically illustrates Type I conventional dendritic cells (cDC1) in anti-tumor immunity.

FIG. 7A-B illustrate how cDC1 plays a critical role in cancer immune control, with primary focus on natural killer (NK) cells:

FIG. 7A graphically illustrates Overall Survival (OS) as a function of time for high and low cDC1 expression (a function of high and low cDC1 gene expression signature); and

FIG. 7B graphically illustrates data showing that NK cells stimulate recruitment of cDC1 into the tumor microenvironment, thus promoting cancer immune control.

FIG. 8 illustrates generation of an exemplary Xcl1 overexpression construct, having the following parameters:

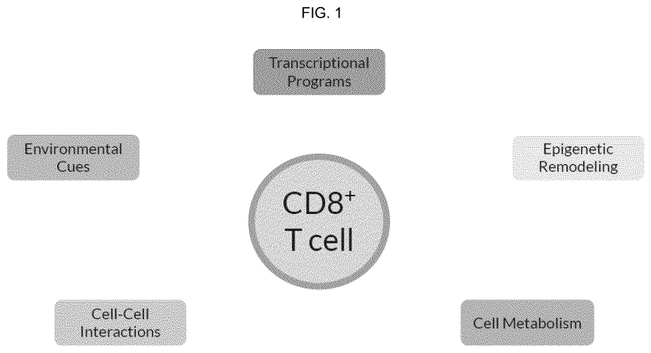

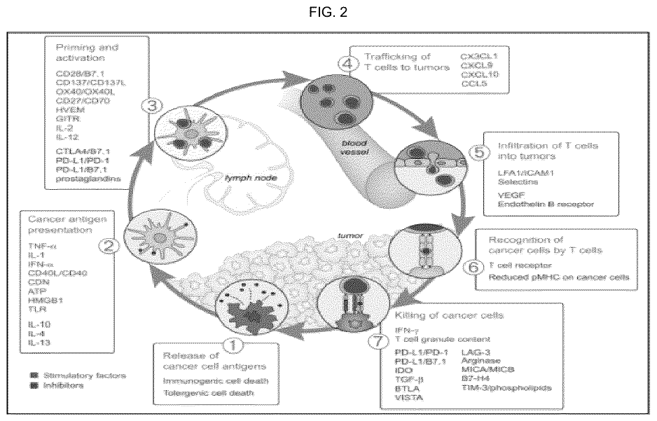

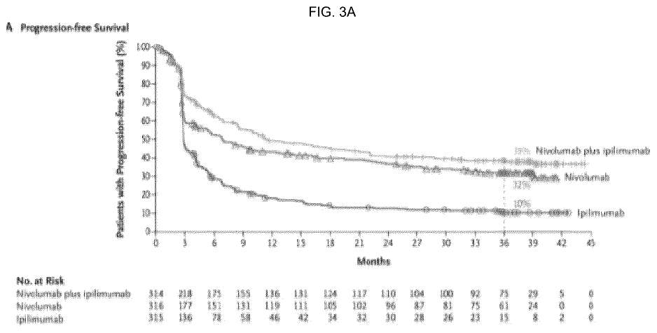

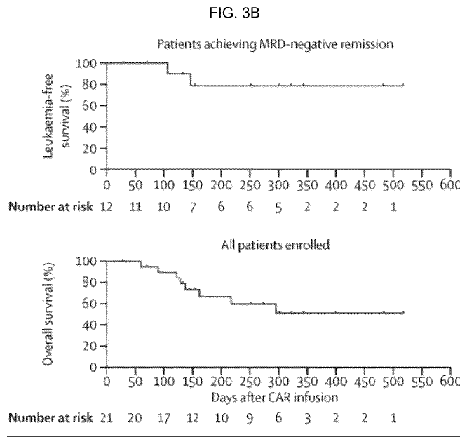

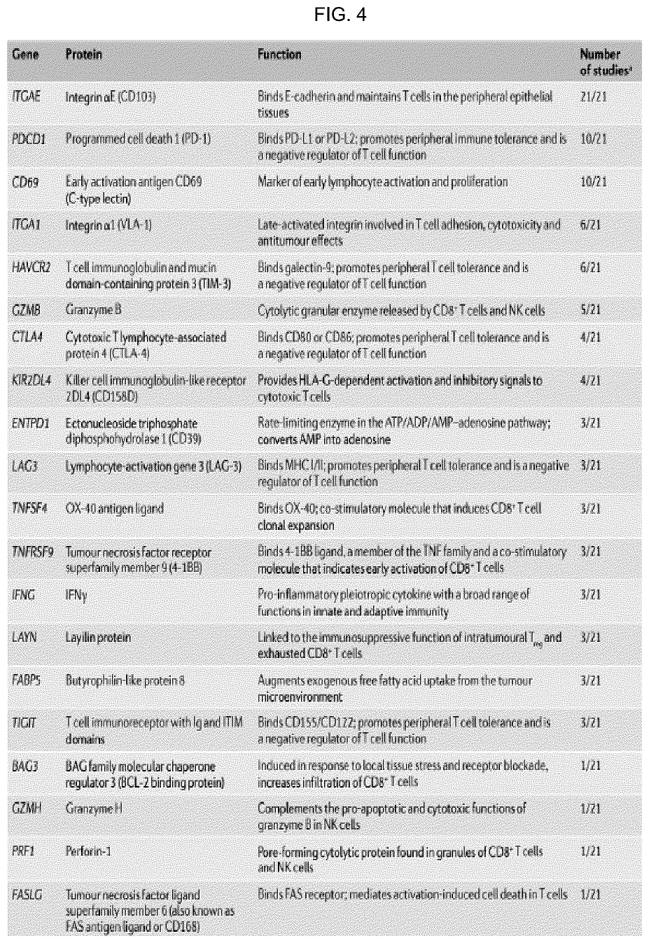

### Component Fragments

| Name | Length | Produced by | 5′ End | 3′ End |
|---|---|---|---|---|
| PMI-Ametrine | 6356 | PCR | Fwd Primer | Rev Primer |
| | | | (auto) | (auto) |
| XCL1 | 345 | Synthetic | — | — |

FIG. 9A-D illustrate data showing that overexpression of XCL1 leads to enhanced tumor control and survival:

FIG. 9A schematically illustrates the exemplary protocol for this study;

FIG. 9B graphically illustrates tumor area as a function of time in days;

FIG. 9C graphically illustrates percentage (%) tumor growth with no T cells, EV (extracellular vesicle) T cells and XCL1 T cells; and

FIG. 9D graphically illustrates probability as a function of time in days, with no T cells, EV (empty vector, control group) T cells and P14 XCL1^{OE} T cells (OE refers to XCL1 overexpression).

FIG. 10A-E illustrate data showing that early expression in cDC1 shifts CD8+ TIL phenotype:

FIG. 10A schematically illustrates the exemplary protocol for this study;

FIG. 10B graphically illustrates percent (%) cDC1 cells in CD8^{EV} and CD8^{XCL1} cells in the tumor (left image) and dLN (right image) (dLN stands for draining lymph nodes);

FIG. 10C graphically illustrates a cell sorting analysis of Tcf1 and Tim3 markers on cells (left image) and percent (%) Tcf1 +Tim3− and Tcf1 +Tim3+ expressing cells (right image), on CD8^{EV} and CD8^{XCL1} cells;

FIG. 10D graphically illustrates a cell sorting analysis of Tcf1 and GzB markers on cells (left image) and percent (%) Tcf1 +GzB − and Tcf1 +GzB+ expressing cells (right image), on CD8^{EV} and CD8^{XCL1} cells; and

FIG. 10E graphically illustrates levels of CD8^{EV} and CD39, PD1 and GzB markers on Tcf1 +Tim3−; Tcf1 −Tim3+, and Tcf1 −Tim3+ expressing cells.

FIG. 11 illustrates an image (left image) of a fluorescent imaging of cells expressing XCR1 (red, or darker staining) and P14 (green, or lighter staining), with a selected subsection of the image expanded (right image).

FIG. 12A-B illustrate data showing that XCL1/XCL2 expression is conserved in human CD8+TIL cells that are: (HPV+/−HNSCC):

FIG. 12A graphically illustrates cell populations having UMAP_2 and UMAP_1 markers; and

FIG. 12B graphically illustrates cell populations having XCR1, XCL1 and XCL2 markers.

FIG. 13 schematically illustrates XCL1 production and XCR1+ recruitment to increase T cell tumor residency and survival.

FIG. 14 schematically illustrates XCL1-CAR T cell design.

FIG. 15 graphically illustrates using CRISPR systems to knock out (KO) SCL1 in CD8+ cells, resulting in LCVM (lymphocytic choriomeningitis virus) and memory T cell differentiation, T_{RM} accumulation, and viral control; and tumor control, TIL phenotype and cDC1 accumulation.

FIG. 16 graphically illustrates using CRISPR systems to knock out (KO) SCL1 in CD8+ cells, resulting in LCVM and memory T cell differentiation, T_{RM} accumulation, and viral control; and tumor control, TIL phenotype and cDC1 accumulation.

FIG. 17 schematically illustrates exemplary methods for leveraging framework of tissue residency to improve CD8 T cells responses in cancer.

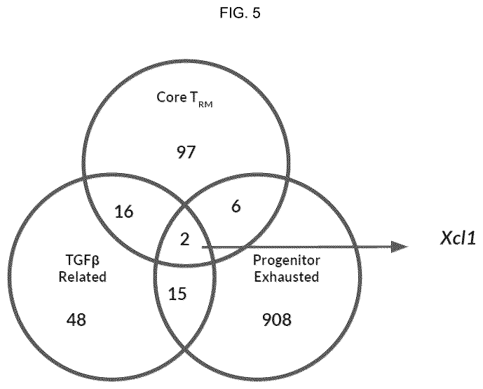

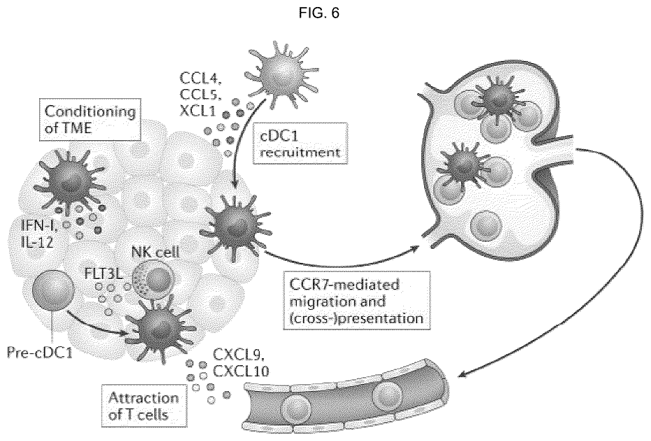

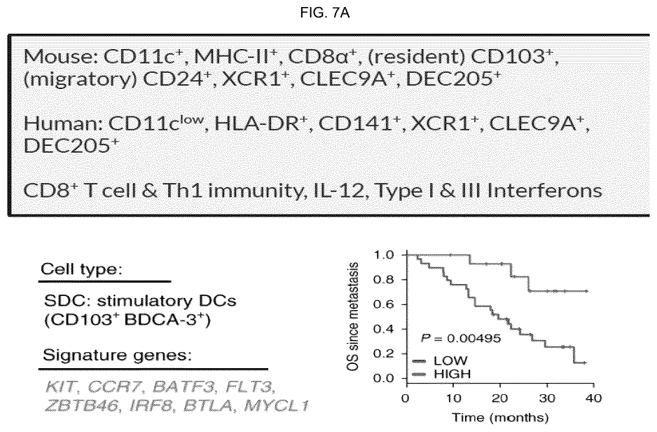

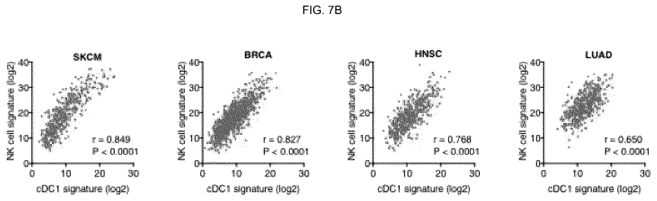

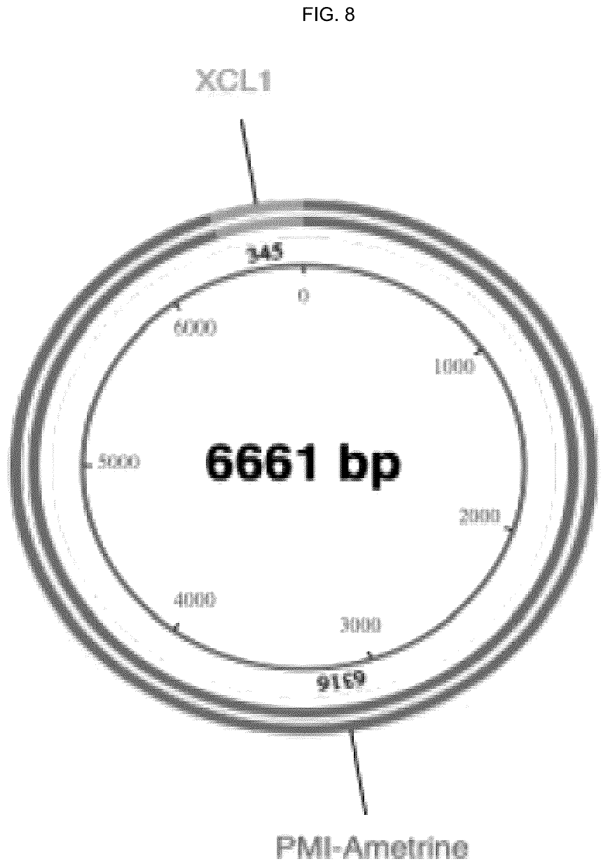

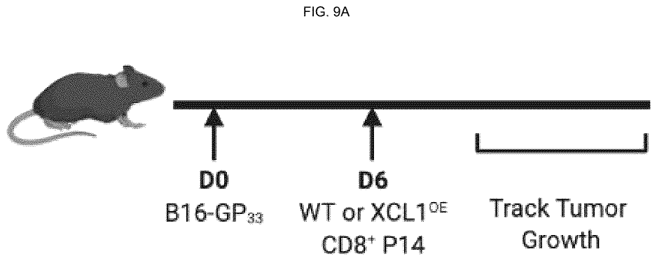

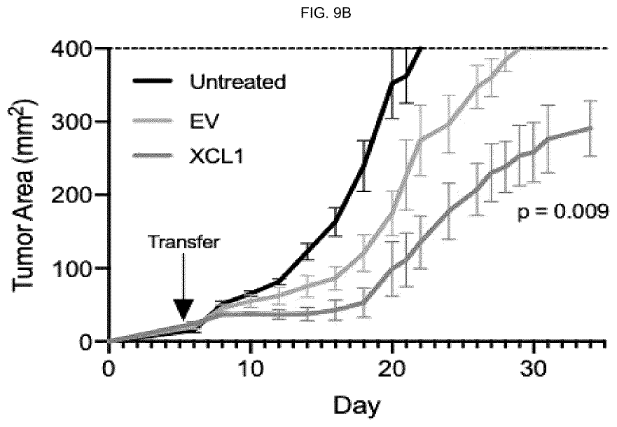

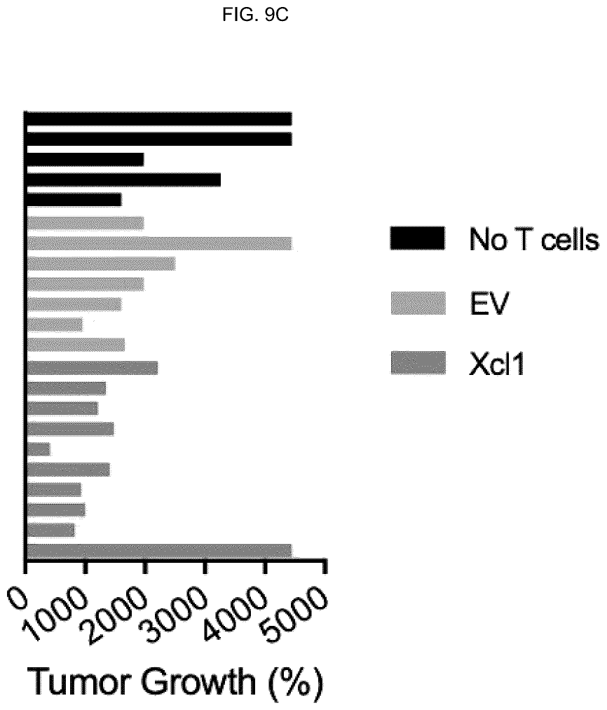

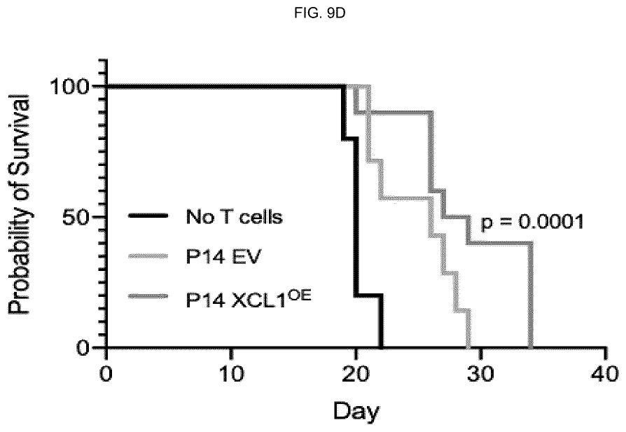

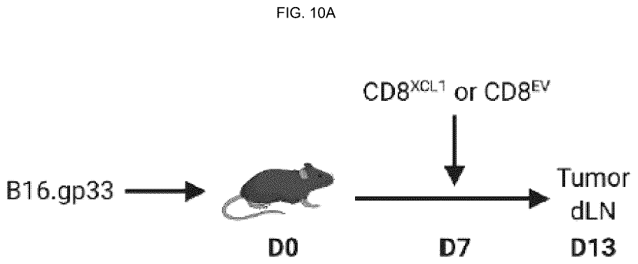

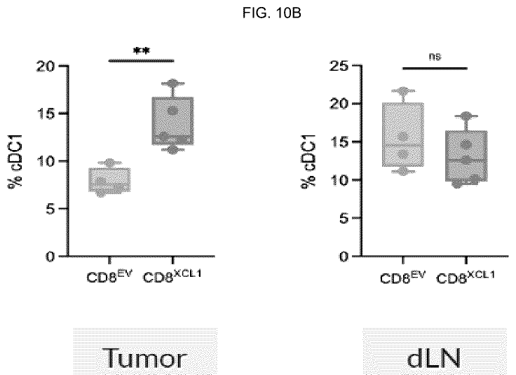

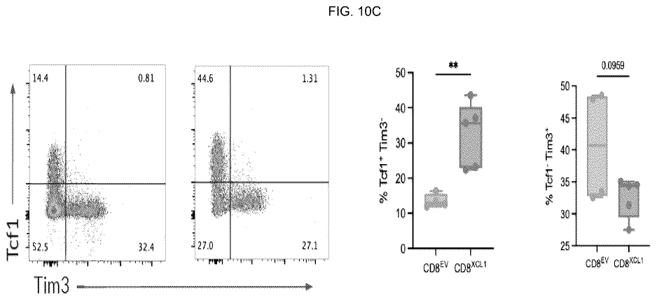

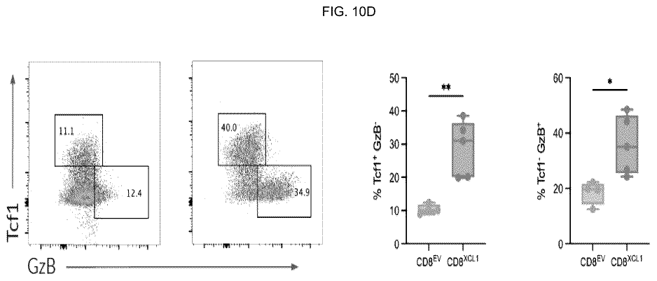

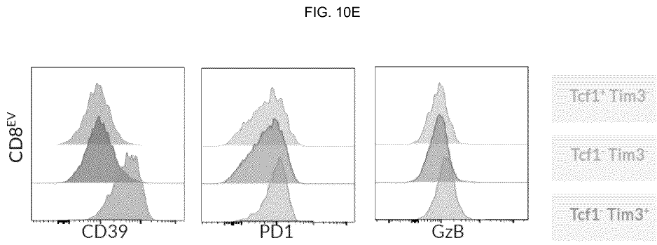

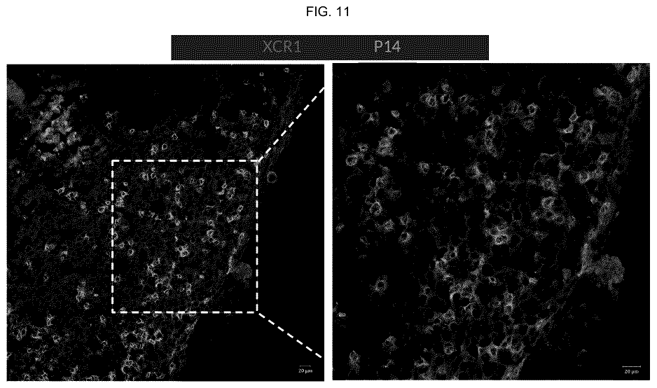

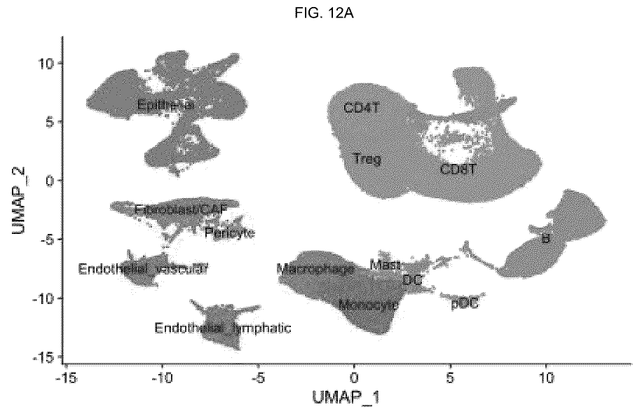

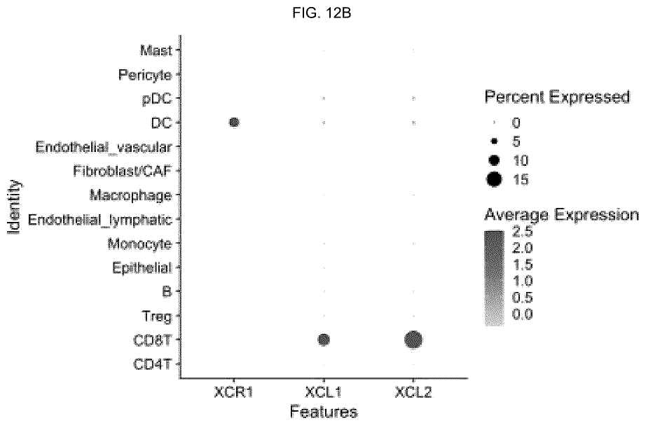

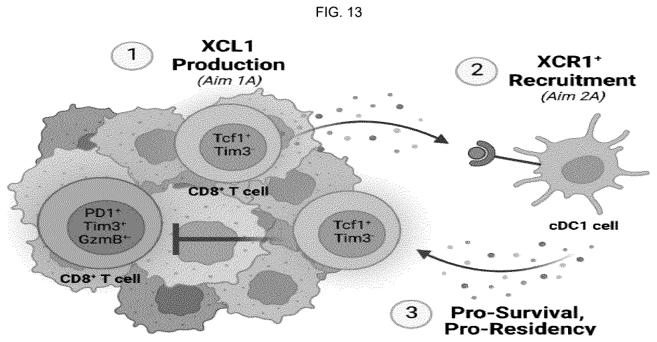

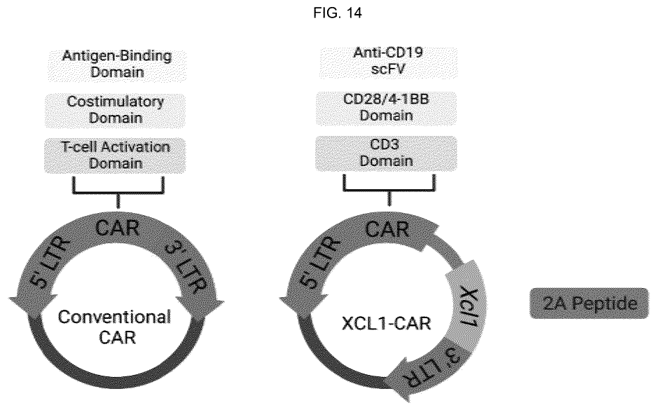

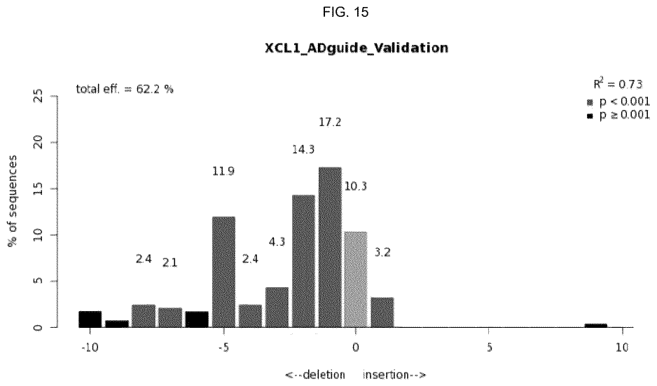

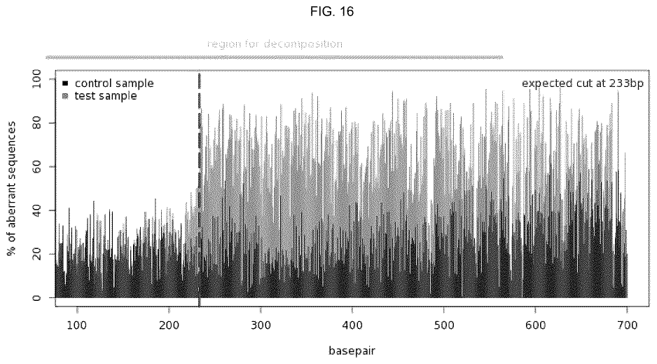

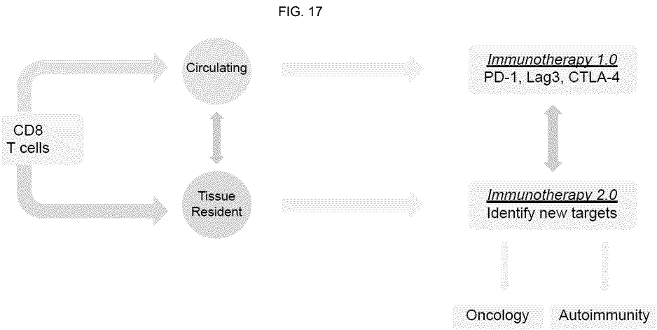

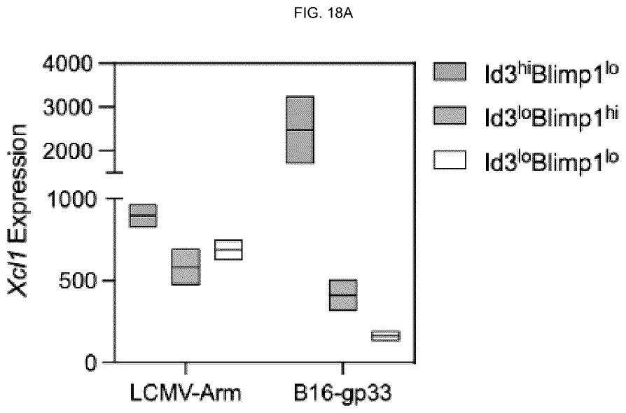

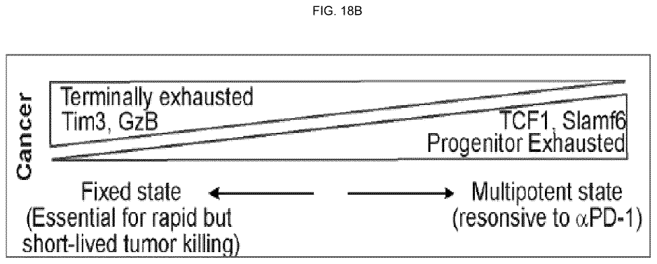

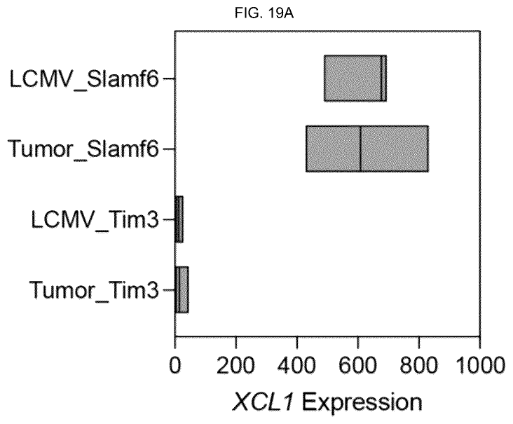

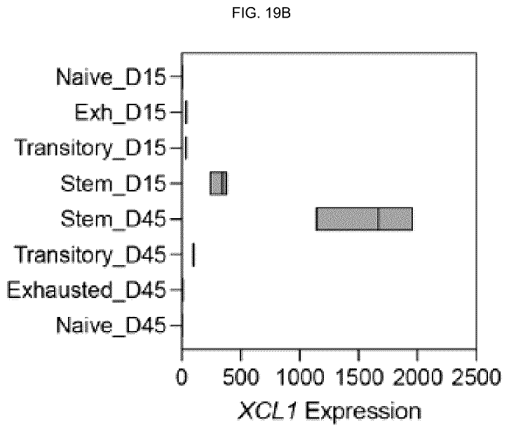

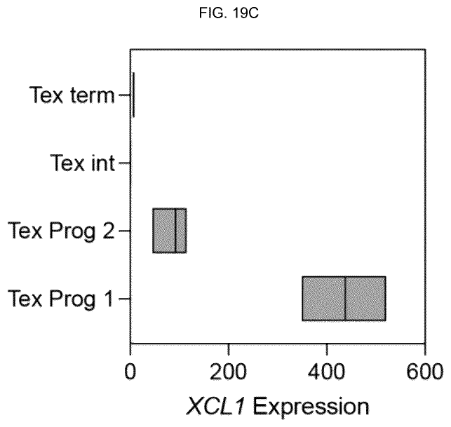

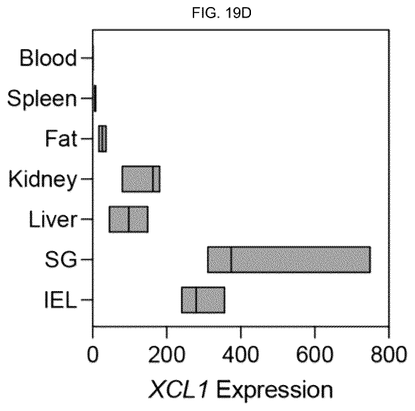

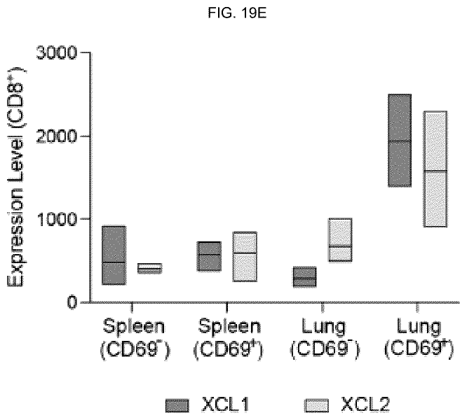

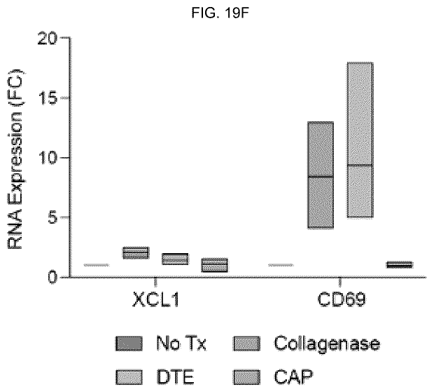

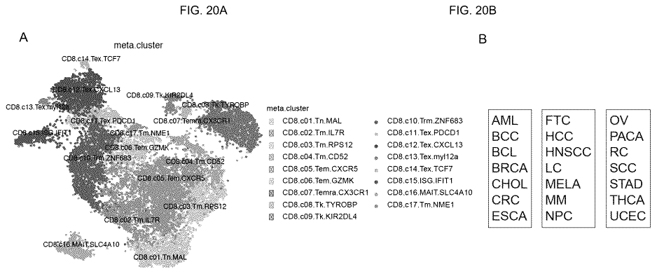

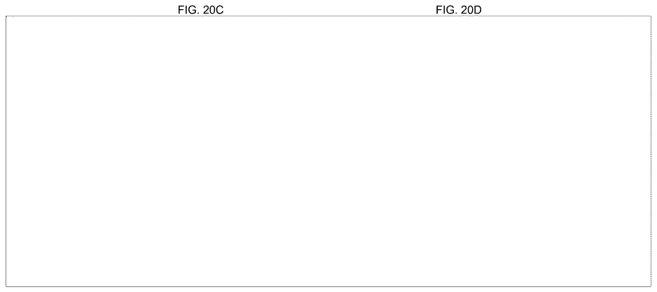

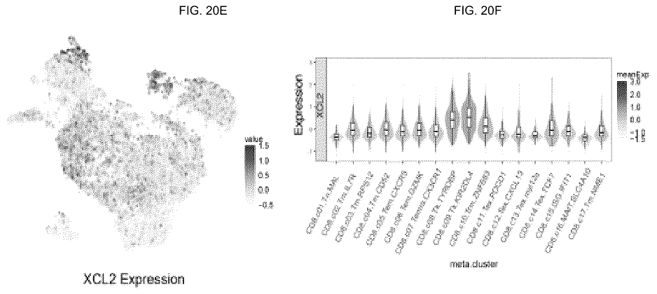

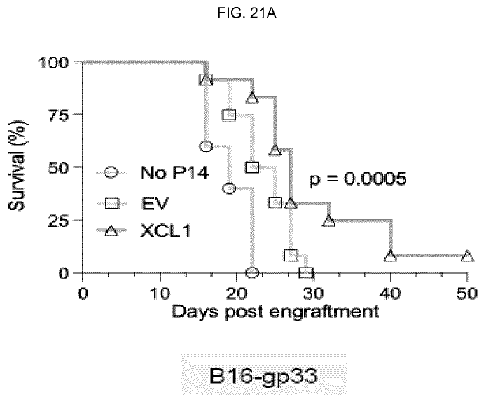

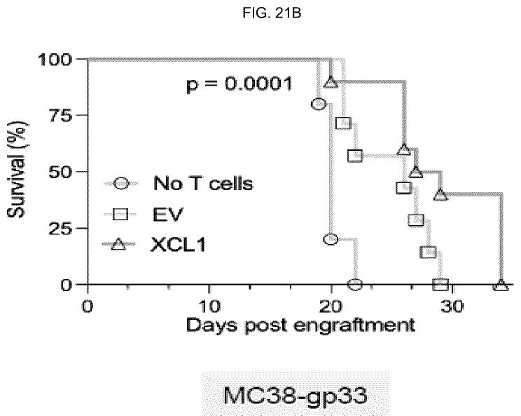

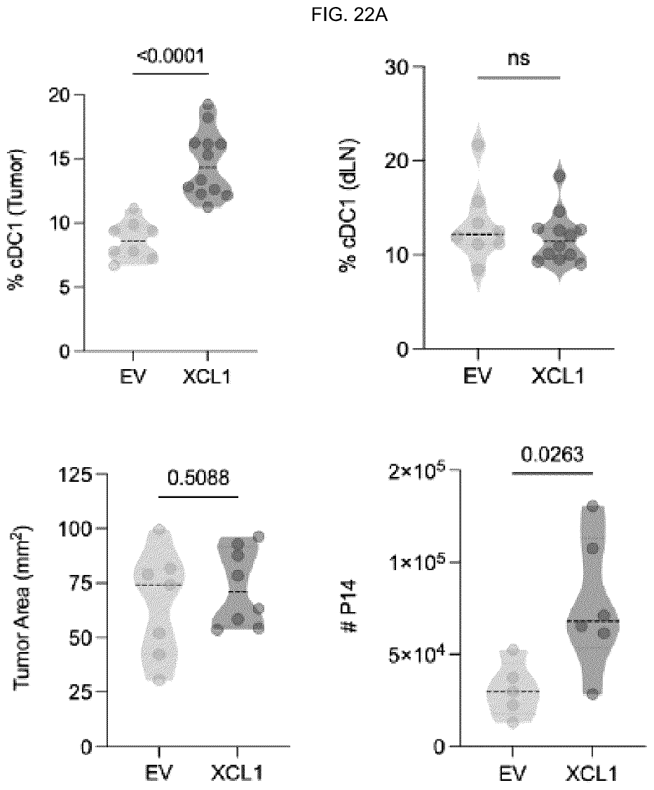

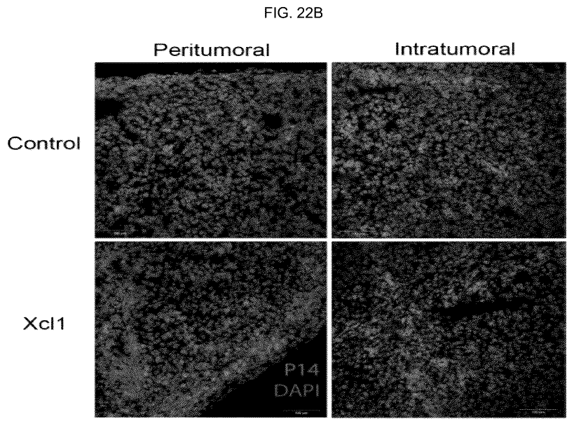

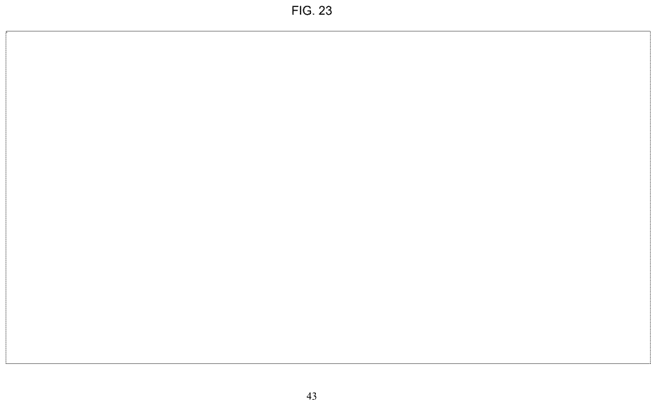

FIG. 18A-B illustrate how CD8+ Tissue Resident Memory CD8+ T cells (T_{RM}) and progenitor-exhausted cells highly express XCL1 (also refer to FIG. 5):

FIG. 18A graphically illustrates XCl1 expression in LCMV-Arm and B16-gp33 cells; and

FIG. 18B schematically illustrates the relationship between terminally exhausted Tim3+, GzB+ cells and TCF1+, Slamf6+ cells, which indicate a fixed physiologic state and a multipotent physiologic state, respectively.

FIG. 19A-F illustrate data showing the expression of XCL1 and XCL2 (human) across mouse and human CD8+ T cell subsets:

FIG. 19A graphically illustrates XCl1 expression in LCMV_Slamf6+, Tumor_Slamf6+, LCMV+Tim3+, and Tumor-Tim3+ cells;

FIG. 19B graphically illustrates XCl1 expression naïve D15, Exh_D15, transitory_D15, stem_D15, stem_D45, transitory D45, exhausted_D45, and naive_D45 cells;

FIG. 19C graphically illustrates XCl1 expression in Tex term, Tex int, Tex Prog 2 and Tex Prog 1 cells;

FIG. 19D graphically illustrates XCl1 expression in blood, spleen, fat, kidney, liver, SG and IEL cells;

FIG. 19E graphically illustrates CD8+ expression with XC11 and XCl2 expression in spleen (CD69⁻), spleen (CD69+), lung (CD69+); and

FIG. 19F graphically illustrates RNA expression with XC11 and CD69 expression in cells fractions: no treatment (tx), DTE tx, collagenase tx, and CAP tx.

FIG. 20A-C illustrate human XCL1 and XCL2 expression across pan-cancer T cell subsets:

FIG. 20A-F illustrate data showing that human XCL1 and XCL2 expression span cancer T cell subsets:

FIG. 20A illustrates an image of a meta-cluster for CD8+ expression, and FIG. 20B shows and summarizes the corresponding data;

FIG. 20C illustrates an image of a meta-cluster for human XCL1 expression, and FIG. 20D graphically summarizes the corresponding data; and

FIG. 20E illustrates an image of a meta-cluster for human XCL2 expression, and FIG. 20F graphically summarizes the corresponding data;

FIG. 21A-B graphically illustrate enforced expression of XCL1 enhances anti-tumor immunity:

FIG. 21A graphically illustrates percent (%) survival as a function of post engraftment for B26-gp33+ cells with: no P14, EV and XCL1; and

FIG. 21B graphically illustrates percent (%) survival as a function of post engraftment for MC38-gp33+ cells with: no T cells, EV and XCL1.

FIG. 22 graphically illustrate data showing that XCL1 enhances intra-tumoral cDC1 accumulation & CD8 T cell trafficking:

FIG. 22A graphically illustrates % cDC1 (tumor) (upper left image), % cDC1 (cLN) (upper right image), tumor area and number of (#) P14 in both EV and XCL1; and

FIG. 22B illustrates an image of control- and XCL1-stained peritumoral and intra-tumoral cells.

FIG. 23 illustrates data showing that antigen-specific CD8+TIL form contacts with XCR1+cDC1: figure illustrates an image of XCR1+ and P14+ stained cells (left image), with the boxed insert magnified (right image).

FIG. 24A illustrates ATAC-seq tracks at XCL1 locus in CD8+T_{RM} populations on left image and on right image the predicted transcription factor binding at differentially accessible peak (highlighted in gray) using (software tool X);

FIG. 24B illustrates a schematic outlining in vitro T_{RM} differentiation assay; and

FIG. 24C illustrates bulk RNA-seq of in vitro generated T_{RM} measuring Itgae (left graph) and XCL1 (right graph) gene expression.

FIG. 25 illustrates TCGA data analysis (GEPIA 2.0) showing melanoma (SKCM) patient survival according to XCL1 & XCL2 expression (median cutoff), the dotted line represents 95% confidence interval, p-value and hazard ratio shown.

FIG. 26 illustrates the top twenty genes with similar expression profile in SKCM as XCL1 (GEPIA 2.0), genes of interest highlighted (shaded).

Like reference symbols in the various drawings indicate like elements.

### DETAILED DESCRIPTION

In alternative embodiments, provided are compositions, including compositions, products of manufacture and kits, and methods, for manipulating, and in particular, augmenting or increasing, T cell activity in vitro and in vivo, by for example increasing XCL1 gene expression in T cells in vivo, or by increasing the number of XCL1 expressing T cells in vivo, or by administering to an individual in need thereof. T cells (such as chimeric antigen receptor (CAR)-T cells) genetically manipulated to have increased XCL1 polypeptide expression.

In alternative embodiments, compositions, products of manufacture and kits, and methods as provided herein are directed to the modulation of, and in particular, increasing the expression of, the chemokine gene XCL1 in T cells for therapeutic applications.

The XCL1 gene is highly expressed by T cell subpopulations of interest, including Tissue Resident Memory CD8+ T cells (T_{RM}) and Progenitor Exhausted CD8+ T cells. T_{RM} are a recently described subset which exhibit organ-specific and conserved adaptations that enable rapid responses upon encountering pathogens and malignant cells. Progenitor exhausted cells require the transcription factor Tcf1, retain polyfunctionality and proliferate upon anti-PD1 treatment. The cognate receptor of XCL1 is XCR1, which is selectively expressed by Type I conventional dendritic cells (cDC1). These cells are critical for cross-presenting antigen to CD8+ T cells and are associated with enhanced anti-tumor immunity. Given the immunosuppressive nature of the tumor microenvironment (TME), modulation of multiple, anti-tumor cell types represents an opportunity to favorably remodel the TME. Each of these populations are highly relevant in the immune response to cancer, so XCL1 gene regulation and increase in XCL1 gene expression can provide new methods of improving T cell and dendritic cell function and design of novel immunotherapies.

In alternative embodiments, provided are methods for regulating (or increasing) XCL1 expression and production by CD8+ T cells. In alternative embodiments, methods as provided herein incorporate the XCL1 gene into CAR-T constructs and deliver these constructs into CAR-T cells. In alternative embodiments, methods as provided herein provide for the targeted delivery of lipid nanoparticles (LNP) containing mRNA encoding for XCL1, and in vivo and in vitro delivery of these LNPs to T cells.

In alternative embodiments, methods as provided herein provide for ex vivo manipulation of (including increase in expression or activity of) XCL1 by T cells, and in alternative embodiments these T cells are administered in vivo for therapeutic treatments, for example, for improving or augmenting an anti-tumor or anti-cancer immune response.

In alternative embodiments, methods as provided herein comprise genetic engineering of T cells to increase XCL1 expression effectively recruits XCR1+ conventional type 1 DCs (dendritic cells) (cDC1s) cells to improve an immune response, for example, to improve or augment an anti-tumor or anti-cancer immune response.

While XCL1 has been shown to efficiently recruit XCR1+cDC1 cells, leveraging T-cell derived XCL1 to improve the immune response has not been previously been exploited. Described here for the first time is data demonstrating the therapeutic efficacy of increased XCL1 expression, or overexpression XCL1 by CD8+ T cells, in cancer treatment (for example, enhanced patient survival). We have shown that T cell recruitment of XCR1+cDC1 positively influences T cell function and differentiation. Accordingly, in alternative embodiments provided herein is a new immunotherapy strategy comprising increased XCL1 expression, or overexpression XCL1. In alternative embodiments, methods as provided herein use multiple approaches for enhancing XCL1 expression by T cells.

In alternative embodiments, methods as provided herein are an immune-therapy that can targets a single cell type to improve immune responses. An advantage of this system is the exploitation of a highly desirable ‘immune cell circuit’ of cDC1 and CD8 T cells via manipulation of the XCL1 chemokine. The system leads to effective recruitment of cDC1 into tumors and also has positive downstream effects on T cell differentiation and function.

In alternative embodiments, methods as provided herein use multiple approaches for increasing XCL1 expression in T cells for therapeutic applications. In alternative embodiments, methods comprise incorporation of an XCL1 gene cassette into a CAR-T genetic construct, which can be delivered into the T cell either in vivo or ex vivo. In alternative embodiments this enables simultaneous expression of synthetic CAR receptor and XCL1 to boost efficacy of an immunotherapy.

In alternative embodiments, another approach comprises the formation of lipid nanoparticles (LNP) containing mRNA encoding XCL1; these may or may not be targeted to T cells via targeting moieties on the LNP surface; this increases XCL1 expression by T cells. In alternative embodiments, the genetically manipulated T cells are delivered systemically in vivo to increase XCL1 expression by T cells in vivo. In alternative embodiments, a construct or an mRNA encoding a dual XCL1/CAR receptor is delivered to the T cell.

While the invention is not limited by any particular mechanism of action, in vivo delivery of XCL1-expressing nucleic acids to T cells, or, in vivo delivery of T cells (such as CAR-T cells) expressing, or overexpressing, XCL1, enables T cells to effectively recruit XCR1+cDC1 cells to improve an immune response, for example, an anti-cancer immune response.

In alternative embodiments therapeutic options include ex vivo manipulation of T cells to express CARs as well as XCL1; additionally, in vivo approaches include targeted nanoparticle-based injections incorporating mRNA for XCL1.

### Formulations and Pharmaceutical Compositions

In alternative embodiments, provided are pharmaceutical formulations or compositions comprising nucleic acids and polypeptides for practicing methods and uses as provided herein to treat, ameliorate, protect against, reverse or decrease the severity or duration of a tumor or a cancer, the methods comprising upregulating or increasing the expression of XCL1 in vivo. In alternative embodiments, provided are pharmaceutical formulations or compositions for use in in vivo, in vitro or ex vivo methods to enhance an immune response, or for recruiting XCR1+ conventional type 1 DC (dendritic cell) (cDC1) cells to improve or enhance an immune response, wherein optionally the immune response is an anti-cancer immune response.

In alternative embodiments, pharmaceutical compositions and formulations used to practice methods and uses as provided herein comprise XCL1-expressing nucleic acids and polypeptides or result in an increase in expression or activity of XCL1-expressing nucleic acids and polypeptides are administered to an individual in need thereof in an amount sufficient to treat, prevent, reverse and/or ameliorate a tumor or cancer. In alternative embodiments, pharmaceutical compositions and formulations used to practice methods and uses as provided herein comprise XCL1-expressing nucleic acids and XCL1 polypeptides or result in an increase in expression or activity of anti-cancer dendritic cells are administered to an individual in need thereof in an amount sufficient to treat, ameliorate, protect against, reverse or decrease the severity or duration a cancer or tumor.

In alternative embodiments, the pharmaceutical compositions used to practice methods and uses as provided herein can be administered intramuscularly (IM), parenterally, topically, orally or by local administration, such as by aerosol or transdermally, or intravitreal injection. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”).

For example, in alternative embodiments, these compositions used to practice methods and uses as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods or uses as provided herein can be in forms such as tablets, pills, powders, capsules, liquids, gels, syrups, slurries, suspensions, etc., suitable for in vivo, in vitro or ex vivo applications.

In alternative embodiments, formulations and pharmaceutical compositions used to practice methods and uses as provided herein can comprise a solution of compositions (which include peptidomimetics, racemic mixtures or racemates, isomers, stereoisomers, derivatives and/or analogs of compounds) disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose, any fixed oil can be employed including synthetic mono- or diglycerides, or fatty acids such as oleic acid. In one embodiment, solutions and formulations used to practice methods and uses as provided herein are sterile and can be manufactured to be generally free of undesirable matter. In one embodiment, these solutions and formulations are sterilized by conventional, well known sterilization techniques.

The solutions and formulations used to practice methods and uses as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compositions and formulations used to practice methods and uses as provided herein can be delivered by the use of liposomes. By using liposomes, particularly where the liposome surface carries ligands specific for target cells (for example, a cancer cell), or are otherwise preferentially directed to a specific tissue or organ type, one can focus the delivery of the active agent into a target cells in an in vivo, in vitro or ex vivo application.

### Nanoparticles, Nanolipoparticles and Liposomes

Also provided are nanoparticles, nanolipoparticles, vesicles and liposomal membranes comprising compounds used to practice methods and uses as provided herein, for example, to deliver compositions comprising XCL1-expressing nucleic acids and XCL1 polypeptides in vivo, for example, to a solid tumor, lymphoid tissue, or for intramuscular injection. In alternative embodiments, these compositions are designed to target specific molecules, including biologic molecules, such as polypeptides, including cell surface polypeptides, for example, for targeting a desired cell type or organ, for example, a cancer cell or lymphoid tissue.

Provided are multilayered liposomes comprising compounds used to practice methods and uses as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods and uses as provided herein.

Liposomes can be made using any method, for example, as described in Park, et al., U.S. Pat. Pub. No. 20070042031, including method of producing a liposome by encapsulating an active agent (for example, XCL1-expressing nucleic acids), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods and uses as provided herein comprise a substituted ammonium and/or polyanions, for example, for targeting delivery of a compound (for example, XCL1-expressing nucleic acid) to a desired cell type (for example, tumor, or lymphoid tissue), as described for example, in U.S. Pat. Pub. No. 20070110798.

Provided are nanoparticles comprising compounds (for example, XCL1-expressing nucleic acid used to practice methods provided herein) in the form of active agent-containing nanoparticles (for example, a secondary nanoparticle), as described, for example, in U.S. Pat. Pub. No. 20070077286. In one embodiment, provided are nanoparticles comprising a fat-soluble active agent or a fat-solubilized water-soluble active agent to act with a bivalent or trivalent metal salt.

In one embodiment, solid lipid suspensions can be used to formulate and to deliver compositions used to practice methods and uses as provided herein to mammalian cells in vivo, for example, to the CNS, as described, for example, in U.S. Pat. Pub. No. 20050136121.

### Delivery Vehicle Modifications and Modification of XCL1-Expressing Nucleic Acid

In alternative embodiments, XCL1-expressing nucleic acid, or XCL1-expressing nucleic acid-comprising nanoparticles, liposomes and the like (for example, comprising or having contained therein XCL1-expressing nucleic acid used to practice methods provided herein) are modified to facilitate IM or any in vivo injections. For example, in alternative embodiments, XCL1-expressing nucleic acid-comprising nanoparticles, liposomes and the like, are engineered to comprise a moiety that allows the XCL1-expressing nucleic acid-comprising nanoparticles, liposomes and the like, to bind to a receptor or cell membrane structure that facilitates delivery into or to a desired cell or organ or tissue. For example, conjugation of mannose-6-phosphate moieties allows the XCL1-expressing nucleic acid-comprising nanoparticles, liposomes and the like, to be taken up by a CNS cell that expresses a mannose-6-phosphate receptor. In alternative embodiments, any protocol or modification of the XCL1-expressing nucleic acid-comprising nanoparticles, liposomes and the like, that facilitates entry or delivery into the CNS or brain in vivo can be used, for example, as described in U.S. Pat. No. 9,089,566.

### Delivery Cells and Delivery Vehicles

In alternative embodiments, any delivery vehicle can be used to practice the methods or uses as provided herein, for example, to deliver compositions (for example, XCL1-expressing nucleic acids) in vivo. For example, delivery vehicles comprising polycations, cationic polymers and/or cationic peptides, such as polyethyleneimine derivatives, can be used for example as described, for example, in U.S. Pat. Pub. No. 20060083737. In one embodiment, a delivery vehicle is a transduced cell engineered to express or overexpress and then secrete an endogenous or exogenous XCL1-expressing nucleic acid.

In one embodiment, a dried polypeptide-surfactant complex is used to formulate a composition used to practice methods as provided herein, for example as described, for example, in U.S. Pat. Pub. No. 20040151766.

In one embodiment, a composition used to practice methods and uses as provided herein can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, cells that will be subsequently delivered into a tumor or lymphoid tissue are transfected or transduced with an XCL1-expressing nucleic acids, for example, a vector, for example, by electro-permeabilization, which can be used as a primary or adjunctive means to deliver the composition to a cell, for example, using any electroporation system as described for example in U.S. Pat. Nos. 7,109,034; 6,261,815; 5,874,268.

In alternative embodiments, XCL1-expressing nucleic acid used to practice embodiments as provided herein comprise or are comprised of human XCL1 cDNA sequence (SEQ ID NO:1):

ATGAGGCTGCTGATCCTGGCCCTGCTGGGCATCTGCAGCCTGACCGCCT
ACATCGTGGAGGGCGTGGGCAGCGAGGTGAGCGACAAGAGGACCTGCGT
GAGCCTGACCACCCAGAGGCTGCCCGTGAGCAGGATCAAGACCTACACC
ATCACCGAGGGCAGCCTGAGGGCCGTGATCTTCATCACCAAGAGGGGCC
TGAAGGTGTGCGCCGACCCCCAGGCCACCTGGGTGAGGGACGTGGTGAG
GAGCATGGACAGGAAGAGCAACACCAGGAACAACATGATCCAGACCAAG
CCCACCGGCACCCAGCAGAGCACCAACACCGCCGTGACCCTGACCGGCT
AG

or a nucleic acid having between about 90% to 99% sequence identity to SEQ ID NO:1 and encoding a polypeptide having XCL1 chemokine activity,
and optionally the XCL1-expressing nucleic acid comprises a nucleic acid sequence that encodes the polypeptide:

(SEQ ID NO: 2)
MRLLILALLGICSLTAYIVEGVGSEVSDKRTCVSLTTQRLPVSRIKTYT
ITEGSLRAVIFITKRGLKVCADPQATWVRDVVRSMDRKSNTRNNMIQTK
PTGTQQSTNTAVTLTG

or a polypeptide having between about 90% to 99% sequence identity to SEQ ID NO:2, or a polypeptide having about 92%, 95% or 97% sequence identity to SEQ ID NO:2 and having XCL1 chemokine activity,
wherein optionally the sequence identities (SEQ IDs) are determined by analysis with a sequence comparison algorithm or by a visual inspection, and optionally the sequence comparison algorithm is a BLAST version 2.2.2 algorithm where a filtering setting is set to blastall-p blastp-d “nr pataa”-F F, and all other options are set to default.

Various sequence comparison programs identified in this patent specification are particularly contemplated for use in this embodiment. Protein and/or nucleic acid sequence homologies may be evaluated using any of the variety of sequence comparison algorithms and programs known in the art. Such algorithms and programs include, but are by no means limited to, TBLASTN, BLASTP, FASTA, TFASTA and CLUSTALW (see, e.g., Pearson and Lipman, Proc. Natl. Acad. Sci. USA 85 (8):2444-2448, 1988; Altschul et al., J. Mol. Biol. 215(3):403-410, 1990; Thompson Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol 48:443, 1970, by the search for similarity method of person & Lipman, Proc. Nat'l. Acad. Sci. USA 85:2444, 1988, by computerized implementations of these algorithms (GAP™, BESTFIT™, FASTA and TFASTA in the Wisconsin Genetics Software Package, Genetics Computer Group, 575 Science Dr., Madison, WI), or by manual alignment and visual inspection. Other algorithms for determining homology or identity include, for example, in addition to a BLAST program (Basic Local Alignment Search Tool at the National Center for Biological Information), ALIGN™, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN™, Las Vegas algorithm, FNAT (Forced Nucleotide Alignment Tool), FRAMEALIGN™ FRAMESEARCH™, DYNAMIC™, FILTER™, FSAP™ (Fristensky Sequence Analysis Package), GAP (Global Alignment Program), GENAL™, GIBBS™ GENQUEST™, ISSC™ (Sensitive Sequence Comparison), LALIGN™ (Local Sequence Alignment), LCP™ (Local Content Program), MACAW™ (Multiple Alignment Construction & Analysis Workbench), MAP (Multiple Alignment Program), MBLKP™, MBLKN™, PIMA™ (Pattern-Induced Multi-sequence Alignment), SAGA™ (Sequence Alignment by Genetic Algorithm) and WHAT-IF™ Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995).

At least twenty-one other genomes have already been sequenced, including, for example, M. genitalium (Fraser et al., 1995), M. jannaschii (Bult et al., 1996), H. influenzae (Fleischmann et al., 1995), E. coli (Blattner et al., 1997) and yeast (S. cerevisiae) (Mewes et al., 1997) and D. melanogaster (Adams et al., 2000). Significant progress has also been made in sequencing the genomes of model organism, such as mouse, C. elegans and Arabadopsis sp. Several databases containing genomic information annotated with some functional information are maintained by different organizations and may be accessible via the internet.

One example of a useful algorithm is BLAST and BLAST 2.0 algorithms, which are described in Altschul et al., Nuc. Acids Res. 25:3389-3402, 1977 and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3 and expectations (E) of 10 and the BLOSUM62 scoring matrix (see Henikoff & Henikoff, Proc. Natl. Acad. Sci. USA 89:10915, 1989) alignments (B) of 50, expectation (E) of 10, M=5, N=−4 and a comparison of both strands.

The BLAST algorithm also performs a statistical analysis of the similarity between two sequences (see, e.g., Karlin & Altschul, Proc. Natl. Acad. Sci. USA 90:5873, 1993). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more in one aspect less than about 0.01 and most in one aspect less than about 0.001.

In one aspect, protein and nucleic acid sequence homologies are evaluated using the Basic Local Alignment Search Tool (“BLAST”) In particular, five specific BLAST programs are used to perform the following task:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) TBLASTN compares a query protein sequence against a nucleotide sequence database translated in all six reading frames (both strands); and
(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is in one aspect obtained from a protein or nucleic acid sequence database. High-scoring segment pairs are in one aspect identified (i.e., aligned) by means of a scoring matrix, many of which are known in the art. In one aspect, the scoring matrix used is the BLOSUM62 matrix (Gonnet (1992) Science 256:1443-1445; Henikoff and Henikoff (1993) Proteins 17:49-61). Less in one aspect, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation). BLAST programs are accessible through the U.S. National Library of Medicine.

The parameters used with the above algorithms may be adapted depending on the sequence length and degree of homology studied. In some aspects, the parameters may be the default parameters used by the algorithms in the absence of instructions from the user.

### In Vivo Delivery of XCL1-Encoding Nucleic Acids

In alternative embodiments, provided are compositions and methods for delivering XCL1-encoding nucleic acids, or vectors or recombinant viruses having contained therein these nucleic acids. In alternative embodiments, the nucleic acids, vectors or recombinant viruses are designed for in vivo or CNS delivery and expression.

In alternative embodiments, provided are compositions and methods for the delivery and controlled expression of an XCL1-encoding nucleic acid or gene, or an expression vehicle (for example, vector, recombinant virus, and the like) comprising (having contained therein) an XCL1-encoding nucleic acid or gene, that results in an XCL1 protein being released into the bloodstream or general circulation where it can have a beneficial effect on in the body, for example, such as the CNS, brain or other targets.

In alternative embodiments, the provided are methods for being able to turn on and turn off XCL1-encoding nucleic acids or gene expression easily and efficiently for tailored treatments and insurance of optimal safety.

In alternative embodiments, XCL1 protein or proteins expressed by the XCL1-encoding nucleic acids or gene(s) have a beneficial or favorable effects (for example, therapeutic or prophylactic) on a tissue or an organ, for example, an anti-cancer effect or immune stimulating effect, even though secreted into the blood or general circulation at a distance (for example, anatomically remote) from their site or sites of action.

In alternative embodiments, provided are expression vehicles, vectors, recombinant viruses and the like for in vivo expression of an XCL1-encoding nucleic acids or gene to practice the methods as provide herein. In alternative embodiments, the XCL1-encoding nucleic acids (such as RNA or DNA), expression vehicles, vectors, recombinant viruses and the like expressing the an XCL1-encoding nucleic acids or gene can be delivered by intravitreal injection or intramuscular (IM) injection (using for example, XCL1-encoding RNA in liposomes), by intravenous (IV) injection, by subcutaneous injection, intrathecal injection, by inhalation, by a biolistic particle delivery system (for example, a so-called “gene gun”), and the like, for example, as an outpatient, for example, during an office visit.

In alternative embodiments, this “peripheral” mode of delivery, for example, expression vehicles, vectors, recombinant viruses and the like injected intravitreal, IM or IV, can circumvent problems encountered when genes or nucleic acids are expressed directly in an organ (for example, a tumor, a lymphoid organ, the brain or into the CNS) itself. Sustained secretion of an XCL1-encoding nucleic acids or XCL1 protein in the bloodstream or general circulation also circumvents the difficulties and expense of administering proteins by infusion.

In alternative embodiments a recombinant virus (for example, a long-term virus or viral vector), or a vector, or an expression vector, and the like, can be injected, for example, in a systemic vein (for example, IV), or by intravitreal, intramuscular (IM) injection, by inhalation, or by a biolistic particle delivery system (for example, a so-called “gene gun”), for example, as an outpatient, for example, in a physician's office. In alternative embodiments, days or weeks later (for example, four weeks later), the individual, patient or subject is administered (for example, inhales, is injected or swallows), a chemical or pharmaceutical that induces expression of the XCL1-encoding nucleic acids or genes; for example, an oral antibiotic (for example, doxycycline or rapamycin) is administered once daily (or more or less often), which will activate the expression of the gene. In alternative embodiments, after the “activation”, or inducement of expression (for example, by an inducible promoter) of the nucleic acid or gene, an XCL1 protein is synthesized and released into the subject's circulation (for example, into the blood), and subsequently has favorable physiological effects, for example, therapeutic or prophylactic, that benefit the individual or patient (for example, benefit heart, kidney or lung function). When the physician or subject desires discontinuation of the XCL1 treatment, the subject simply stops taking the activating chemical or pharmaceutical, for example, antibiotic.

Alternative embodiments comprise use of “expression cassettes” comprising or having contained therein a nucleotide sequence used to practice methods provided herein, for example, an XCL1-encoding nucleic acid, which can be capable of affecting expression of the nucleic acid, for example, as a structural gene or a transcript (for example, encoding an XCL1 protein) in a host compatible with such sequences. Expression cassettes can include at least a promoter operably linked with the polypeptide coding sequence or inhibitory sequence; and, in one aspect, with other sequences, for example, transcription termination signals. Additional factors necessary or helpful in effecting expression may also be used, for example, enhancers.

In alternative aspects, expression cassettes also include plasmids, expression vectors, recombinant viruses, any form of recombinant “naked DNA” vector, and the like. In alternative aspects, a “vector” can comprise a nucleic acid that can infect, transfect, transiently or permanently transduce a cell. In alternative aspects, a vector can be a naked nucleic acid, or a nucleic acid complexed with protein or lipid. In alternative aspects, vectors can comprise viral or bacterial nucleic acids and/or proteins, and/or membranes (for example, a cell membrane, a viral lipid envelope, etc.). In alternative aspects, vectors can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, a vector can be stably replicated by the cells during mitosis as an autonomous structure, or can be incorporated within the host's genome.

In alternative aspects, “promoters” include all sequences capable of driving transcription of a coding sequence in a cell, for example, a mammalian cell such as a retinal cell. Promoters used in the constructs provided herein include cis-acting transcriptional control elements and regulatory sequences that are involved in regulating or modulating the timing and/or rate of transcription of a nucleic acid, for example, an AIBP-encoding nucleic acid. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

In alternative embodiments, “constitutive” promoters can be those that drive expression continuously under most environmental conditions and states of development or cell differentiation. In alternative embodiments, “inducible” or “regulatable” promoters can direct expression of a nucleic acid, for example, an AIBP-encoding nucleic acid, under the influence of environmental conditions, administered chemical agents, or developmental conditions.

### Gene Therapy and Gene Delivery Vehicles

In alternative embodiments, methods of the invention comprise use of nucleic acid (for example, an XCL1-encoding nucleic acid) delivery systems to deliver a payload of the nucleic acid or gene, or XCL1-encoding nucleic acid, transcript or message, to a cell or cells in vitro, ex vivo, or in vivo, for example, as gene therapy delivery vehicles.

In alternative embodiments, expression vehicle, vector, recombinant virus, or equivalents used to practice methods provided herein are or comprise: an adeno-associated virus (AAV), a lentiviral vector or an adenovirus vector; an AAV serotype AAV5, AAV6, AAV8 or AAV9; a rhesus-derived AAV, or the rhesus-derived AAV AAVrh.10hCLN2; an organ-tropic AAV, or a neurotropic AAV; and/or an AAV capsid mutant or AAV hybrid serotype. In alternative embodiments, the AAV is engineered to increase efficiency in targeting a specific cell type that is non-permissive to a wild type (wt) AAV and/or to improve efficacy in infecting only a cell type of interest. In alternative embodiments, the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid. It is well known in the art how to engineer an adeno-associated virus (AAV) capsid in order to increase efficiency in targeting specific cell types that are non-permissive to wild type (wt) viruses and to improve efficacy in infecting only the cell type of interest; see for example, Wu et al., Mol. Ther. 2006 September; 14(3):316-27. Epub 2006 July 7; Choi, et al., Curr. Gene Ther. 2005 June; 5(3):299-310.

For example, in alternative embodiments, serotypes AAV-8, AAV-9, AAV-DJ or AAV-DJ/8™ (Cell Biolabs, Inc., San Diego, CA), which have increased uptake in brain tissue in vivo, are used to deliver an AIBP-encoding nucleic acid payload for expression in the CNS. In alternative embodiments, the following serotypes, or variants thereof, are used for targeting a specific tissue:

| Tissue | Optimal Serotype |
|---|---|
| CNS | AAV1, AAV2, AAV4, AAV5, AAV8, AAV9 |
| Photoreceptor Cells | AAV2, AAV5, AAV8 |
| RPE (Retinal Pigment | AAV1, AAV2, AAV4, AAV5, AAV8 |
| Epithelium) | |
| Skeletal Muscle | AAV1, AAV6, AAV7, AAV8, AAV9 |

In alternative embodiments, the rhesus-derived AAV AAVrh.10hCLN2 or equivalents thereof can be used, wherein the rhesus-derived AAV may not be inhibited by any pre-existing immunity in a human; see for example, Sondhi, et al., Hum Gene Ther. Methods. 2012 October; 23(5):324-35, Epub 2012 Nov. 6; Sondhi, et al., Hum Gene Ther. Methods. 2012 Oct. 17; teaching that direct administration of AAVrh.10hCLN2 to the CNS of rats and non-human primates at doses scalable to humans has an acceptable safety profile and mediates significant payload expression in the CNS.

Because adeno-associated viruses (AAVs) are common infective agents of primates, and as such, healthy primates carry a large pool of AAV-specific neutralizing antibodies (NAbs) which inhibit AAV-mediated gene transfer therapeutic strategies, methods provided herein can comprise screening of patient candidates for AAV-specific NAbs prior to treatment, especially with the frequently used AAV8 capsid component, to facilitate individualized treatment design and enhance therapeutic efficacy; see, for example, Sun, et al., J. Immunol. Methods. 2013 Jan. 31; 387(1-2):114-20, Epub 2012 Oct. 11.

In alternative embodiments, the XCL1-encoding nucleic acid is delivered in vivo using methods as provided herein can be in the form of, or comprise, an RNA, for example, mRNA, which can be formulated in a lipid formulation, in a liposomal vesicle, or a liposome and injected for example intramuscularly (IM), for example using formulations and methods as described in U.S. patent application no. US 20210046173 A1, which describes delivering to a subject (for example, via intramuscular administration) the XCL1-encoding nucleic acid that comprises a RNA (for example, mRNA) that comprises an open reading frame (ORF) that comprises (or consists of, or consists essentially of) or encodes for the XCL1 protien; wherein optionally the RNA (or the DNA-carrying expression vehicle) is formulated in a liposome, or a lipid nanoparticle (LNP), or nanoliposome, that comprises: non-cationic lipids comprise a mixture of cholesterol and 1,2-distearoyl-sn-glycero-3-phosphocholine (DSPC), or a PEG-lipid, or PEG-modified lipid, or LNP, or an ionizable cationic lipid; or a mixture of (13Z,16Z)-N,N-dimethyl-2-nonylhenicosa-12,15-dien-1-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG). In alternative embodiments, the LNP comprises 20-99.8 mole % ionizable cationic lipids, 0.1-65 mole % non-cationic lipids, and 0.1-20 mole % PEG-lipid. In alternative embodiments, the LNP comprises an ionizable cationic lipid selected from the group consisting of (2S)-1-({6-[(3))-cholest-5-en-3-yloxy]hexyl}oxy)-N,N-dimethyl-3-[(9 Z)-octadec-9-en-1-yloxy]propan-2-amine; (13Z,16Z)-N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing. In alternative embodiments, the PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In alternative embodiments, the ionizable cationic lipid comprises: 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy) heptadecanedioate (L319), (13Z,16Z)-N,N-dimethyl-3-nonyldocosa-13,16-dien-1-amine, (12Z,15Z)-N,N-dimethyl-2-nonylhenicosa-12,15-dien-1-amine, and N,N-dimethyl-1-[(1S,2R)-2-octylcyclopropyl]heptadecan-8-amine. In one embodiment, the lipid is (13Z,16Z)-N,N-dimethyl-3-nonyldocosa-13,16-dien-1-amine or N,N-dimethyl-1-[(1S,2R)-2-octylcyclopropyl]heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Dosaging

The pharmaceutical compositions and formulations used to practice methods and uses as provided herein can be administered for prophylactic and/or therapeutic treatments, for example, to treat, ameliorate a cancer or tumor, or enhance an immune response, to recruit XCR1⁺ conventional type 1 DC (dendritic cell) (cDC1) cells to improve an immune response, wherein optionally the immune response is an anti-cancer immune response. In therapeutic applications, compositions are administered to a subject already suffering from a disease, condition, infection or defect in an amount sufficient to cure, alleviate or partially arrest the clinical manifestations of the disease, condition, infection or disease and its complications (a “therapeutically effective amount”), including for example, cancer. For example, in alternative embodiments, XCR1-expressing nucleic acid-comprising pharmaceutical compositions and formulations as provided herein are administered to an individual in need thereof in an amount sufficient to treat, ameliorate, protect against, reverse or decrease the severity of a cancer, or to or recruit XCR1⁺ conventional type 1 DC (dendritic cell) (cDC1) cells to improve an immune response, wherein optionally the immune response is an anti-cancer immune response.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

In alternative embodiments, viral vectors such as adenovirus or AAV vectors are administered to an individual in need therein, and in alternative embodiment the dosage administered to a human comprises: a dose of about 2×10¹² vector genomes per kg body weight (vg/kg), or between about 10¹⁰ and 10¹⁴ vector genomes per kg body weight (vg/kg), or about 10⁹, 10¹⁰, 10¹¹, 10¹², 10¹³, 10¹⁴, 10¹⁵, or more vg/kg, which can be administered as a single dosage or in multiple dosages, as needed. In alternative embodiments, these dosages are administered intravitreally, orally, IM, IV, or intrathecally. In alternative embodiments, the vectors are delivered as formulations or pharmaceutical preparations, for example, where the vectors are contained in a nanoparticle, a particle, a micelle or a liposome or lipoplex, a polymersome, a polyplex or a dendrimer.

In alternative embodiments, these dosages are administered once a day, once a week, or any variation thereof as needed to maintain in vivo expression levels of XCR1-expressing nucleic acid or XCR1 protein, which can be monitored by measuring actually expression of XCR1 or by monitoring of therapeutic effect, for example, to treat, ameliorate, protect against, reverse or decrease the severity of a cancer or recruit XCR1⁺ conventional type 1 DC (dendritic cell) (cDC1) cells to improve an immune response, wherein optionally the immune response is an anti-cancer immune response. The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, alternative exemplary pharmaceutical formulations for oral administration of compositions used to practice methods as provided herein are in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, for example, compositions for treating any neurological or neuromuscular disease, condition, infection or injury, including related inflammatory and autoimmune diseases and conditions, and the like. For example, the methods and/or compositions and formulations as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (for example, a ficolin), carbohydrate-binding domains, and the like and combinations thereof.

### CRISPR Gene Therapy

In alternative embodiments, T cells genetically manipulated or engineered to have increased XCL1 polypeptide expression, wherein optionally the T cell is a chimeric antigen receptor (CAR)-T cell, and optionally the CAR-T cell targets cancer, wherein the T cells can be delivered in vivo or ex vivo, or XCL1 polypeptide-expressing nucleic acids or expression vehicles containing XCL1 polypeptide-expressing nucleic acids, which can be delivered in vivo or ex vivo, are used to practice embodiments as provided herein, and in alternative embodiments, these T cells are manipulated to express or overexpress XCL1 polypeptides using a CRISPR system such as a CRISPR-Cas9 system. For example, in one embodiment, a CRISPR system increases expression of a homologous XCL1 gene, message and/or polypeptide, and in another embodiment a CRISPR system is used to implant a heterologous XCL1-expressing nucleic acid.

Delivery of XCL1 exogenous nucleic acids, Cas9, sgRNA, and associated complexes into cells such as T cells can occur using viral and non-viral systems: for example, electroporation of DNA, RNA, or ribonucleocomplexes; chemical transfection techniques utilizing lipids and peptides (particularly to introduce sgRNAs in complex with Cas9 into cells); nanoparticle-based delivery for transfection, and the like. Some categories of cells are more difficult to transfect, including stem cells, neurons, and hematopoietic cells, and these require more efficient delivery systems, such as those based on lentivirus (LVs), adenovirus (AdV), and adeno-associated virus (AAV).

Variants of CRISPR-Cas9 an be used to allow gene activation or genome editing with an external trigger such as light or small molecules: including photoactivatable CRISPR systems developed by fusing light-responsive protein partners with an activator domain and a dCas9 for gene activation, or by fusing similar light-responsive domains with two constructs of split-Cas9, or by incorporating caged unnatural amino acids into Cas9, or by modifying the guide RNAs with photocleavable complements for genome editing.

In alternative embodiments, “dead” versions of Cas9 (dCas9) are used to eliminate CRISPR's DNA-cutting ability while preserving its ability to target desirable sequences. Various regulatory factors can be added to dCas9s, enabling turning any gene on or off or adjust its level of activity. Like RNAi, CRISPR interference (CRISPRi) can turn off genes in a reversible fashion by targeting, but not cutting a site. The targeted site is methylated, epigenetically modifying the gene. This modification inhibits transcription. These precisely placed modifications may then be used to regulate the effects on gene expression (for example, XCL1 exogenous nucleic acids) and DNA dynamics after the inhibition of certain genome sequences within DNA.

In alternative embodiments, CRISPR-Cas13 fused to deaminases is used to direct mRNA editing; for example, Cas7-11, is better suited for therapeutic RNA editing than Cas13, and enables sufficiently targeted cuts.

In alternative embodiments, any CRISPR system can be used to practice methods as provided herein, for example, as described in US 2022 0387560 A1, which describes methods of treating and/or correcting ocular disease in vivo using an Adeno-associated virus (AAV) system, where the AAV system employs a nucleic acid encoding a CRISPR-Cas9 system for targeted gene disruption or correction; or US 2022 0389398 A1 that describes using engineered CRISPR/Cas effector enzymes, such as Cas13 (Cas13d, Cas13e, or Cas13f) that maintain guide-sequence-specific endonuclease activity and lack guide-sequence-independent collateral endonuclease activity; or US 2023 0029506 A1, which describes therapeutic applications of the crispr-cas systems and compositions for genome editing; or, US 2020 0340012 A1, which describes a modular CRISPR-Cas9 architecture that allows better delivery, specificity and selectivity of gene editing; or U.S. Pat. No. 8,771,945, which describes CRISPR-Cas systems and methods for altering expression of gene products; or WO 2023 283420 A2 which describes therapeutic gene silencing with crispr-cas13.

In alternative embodiments, design of a nucleic acid encoding XCL1 exogenous nucleic acids, or for increasing homologous XCL1 expression, can be based on the nucleic acid sequence of the gene or transcript of human XCL1 (see for example SEQ ID NO:1)

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Exemplary Methods

This example demonstrates exemplary methods used to generate validating data as provided herein, as illustrated for example, in the figures:

### Mice

All mice were bred on the C57BL6/J background and housed in specific pathogen-free conditions in accordance with the Institutional Animal Care and Use Committees of the University of California, San Diego. Both male and female mice were used throughout the study, with sex matched T cell donors and recipients (or female donor cells transferred into male recipients) and between 1.5 and 4 months old. C57BL/6J mice (stock #000664; The Jackson Laboratory), XCR1_{DTR/Venus} (Gutkind Laboratory, UCSD), P14 mice (with transgenic expression of H-2D b-restricted TCR specific for LCMV glycoprotein GP 33-41; stock #037394-JAX; The Jackson Laboratory), CD45.1+, and CD45.1.2+ congenic mice were bred in house.

### T Cell Activation, Transduction

For CD8+ T cell activation, naive CD8+ T cells from spleens and lymph nodes were negatively enriched with LS MACS columns (Miltenyi Biotec) using biotin anti-CD4 (GK1.5), anti-Ter119 (TER-119), anti-GR-1 (RB6-8C5), anti-MHCII (M5/114.15.2), anti B220 (RA3-6B2), and anti-NK 1.1 (PK136). 2×10⁶ P14 cells were plated in a well of a 6-well plate that was pre-coated with 100 μg/ml goat anti-hamster IgG (H+L, Thermo Fisher Scientific). The activation medium contained 1 μg/ml anti-CD3 (145-2C11) and 1 μg/ml anti-CD28 (37.51) (eBioscience). Culture medium was replaced after 18 h of activation with retroviral supernatant mixed with 50 M BME and 8 g/ml polybrene (Millipore) followed by spin-infection (1-hour centrifugation at 2000 RPM, 37 °C). The plate was incubated at 37 °C for 3 hours after spin-infection, and then the retroviral supernatant was replaced by T cell medium supplemented with IL-2 and incubated for 24-72 hours prior to use.

### Retroviral Production

For forced XCL1 experiments, retroviral particles were generated using platinum E cells grown in 10-cm plates with full selection media (DMEM, 10% FBS [v/v], 2 mM L-glutamine, 100 U/mL penicillin-streptomycin, 1 ug/mL puromycin, and 10 μg/mL blasticidin). Eighteen hours before transfection selection media was replaced with antibiotic-free media (DMEM, 10% FBS [v/v], 2 mM L-glutamine). For each 10-cm plate, 10 ug of each construct and 5 μg of pCL-Eco helper plasmids were mixed in Opti-MEM (Thermo Fisher Scientific) to a volume of 700 uL. This was combined with 45 μL of TRANSIT-LT1™ reagent (Mirus Bio) and 655 μL of OPTI-MEM™ for 20 min at room temperature. This mixture was added dropwise to each 10-cm plate. Twelve hours later media was replaced, and viral supernatant was subsequently harvested at 48 and 72 hours. Retroviral supernatant was filtered and stored at −80 for future use.

### Cell Culture

Male B16 melanoma cells and MC38 colorectal tumor cells expressing the LCMV glycoprotein epitope amino acid 33-41 (B16-GP 33-41) and female PLAT-E cells were maintained in DMEM containing 5% bovine growth serum, 1% HEPES and 0.1% 2-Mercaptoethanol. Both cell lines have been confirmed to be free of mycoplasma through qPCR. Retroviral particles were generated in PLAT-E cells as previously described.

### Tumor Studies

B16-GP₃₃₋₄₁ cells and MC38-GP₃₃₋₄₁ (5×10₅) were transplanted subcutaneously into the right flank of wild-type mice. After tumors became palpable, 7-9 days post transplantation, 1-2.5×10₆P14 cells that were transduced with XCL1 or EV and expanded in vitro with 50 U/mL IL-2 for 2-3 days and transferred intravenously. Tumors were monitored daily and mice with ulcerated tumors or tumors exceeding 1500 mm 3 in size were euthanized in accordance with UCSD (university) Institutional Animal Care and Use Committee (IACUC). TILs were isolated as previously described 4-8 days following adoptive transfer.

### Quantification and Statistical Analysis

Statistical parameters are reported in the Figures; asterisks in figures denote statistical significance (*p<0.05, **p<0.01, ***p<0.001, ****p<0.0001), and data is judged to be statistically significant when p<0.05. All sequencing was performed and analyzed independently in at least two biological replicates, and gene expression signatures were compared by Fisher's exact tests. In all other data analysis, statistical significance was calculated by unpaired or paired two-tailed Student's t test. Statistical analysis was performed in GRAPHPAD PRISM™ software and R.

FIG. 19A: illustrates XCL1 gene expression in P14 T cells derived from indicated studies; expression data during LCMV-C113 chronic viral infection and B16 melanoma challenge in indicated subsets;

FIG. 19B illustrates expression data during LCMV-C113 in indicated subsets and timepoints;

FIG. 19C illustrates expression data during LCMV-C113 using alternative gating scheme; and

FIG. 19D illustrates expression data from CD8⁺ T_{RM} during LCMV-Arm.

FIG. 19E: illustrates XCL1 and XCL2 gene expression in human CD8 T cells in indicated subsets.

FIG. 19F: illustrates splenic CD8+ T cell gene expression of XCL1 and CD69 using different tissue digestion methods.

A number of embodiments of the invention have been described. Nevertheless, it can be understood that various modifications may be made without departing from the spirit and scope of the invention. Accordingly, other embodiments are within the scope of the following claims.

1: A method for:
(i) treating, ameliorating, slowing the progression of, decreasing the severity of symptoms of, or preventing a tumor or a cancer,
(ii) stimulating T cell generation or activity, optionally stimulating cancer-targeting T cell generation or activity, or
(iii) recruiting XCR1⁺ conventional type 1 DC (dendritic cell) (cDC1) cells to improve an immune response, wherein optionally the immune response is an anti-cancer immune response,
comprising administering to an individual in need thereof:
(a) a nucleic acid encoding a XCL1, or lymphotactin, polypeptide, wherein optionally the XCL1-expressing nucleic acid is an XCL1 gene or XCL1 mRNA sequence, or the XCL1-expressing nucleic acid is an RNA or a DNA molecule, or a synthetic nucleic acid,
(b) a T cell genetically manipulated or engineered to have increased XCL1 polypeptide expression, or
(c) a bispecific antibody or a bispecific antigen binding polypeptide, wherein the bispecific antibody or the bispecific antigen binding polypeptide can simultaneously specifically bind:
(i) an XCR1 (receptor for XCL1) expressed on an outer surface of a T cell, and
(ii) a T cell receptor (TCR) or CD3 protein complex on the surface of the T cell.

2: The method of claim 1, wherein the XCL1-encoding nucleic acid, the T cell genetically manipulated or engineered to have increased XCL1 polypeptide expression, or the bispecific antibody or a bispecific antigen binding polypeptide, is contained in a liposome, a liposomal vesicle, an expression construct, a plasmid, an expression vehicle, a virus or a vector, and the expression construct, plasmid, expression vehicle, virus or vector is delivered or administered to the individual in need thereof.

3: The method of claim 1, wherein the XCL1-expressing nucleic acid, the expression construct, plasmid, expression vehicle, virus or vector, the bispecific antibody or a bispecific antigen binding polypeptide, or the genetically manipulated or engineered T cell, is formulated:
(a) in a liquid, a gel, a hydrogel, a vesicle, a liposome, a liposomal vesicle, a nanoparticle, a nanolipid particle, a powder or an aqueous or a saline formulation, or for administration in vitro or in vivo;
(b) for enteral or parenteral administration;
(c) in or as a liposome, a nanoparticle, or a nanoliposome;
(d) in or as a tablet, a pill, a capsule, a gel, a hydrogel, a geltab, a liquid, a powder, an emulsion, a lotion, an aerosol, a spray, a lozenge, an aqueous or a sterile or an injectable solution, an eye drop, or an implant; or
(e) for intravenous injection, subcutaneous injection, intramuscular injection, inhalation, intrathecal injection, or intravitreal injection.

4: The method of claim 1, wherein the nucleic acid encoding the XCL1 polypeptide, or the XCL1 gene or XCL1 mRNA sequence, or the XCL1-expressing RNA or DNA nucleic acid, the expression construct, plasmid, expression vehicle, virus or vector, the bispecific antibody or a bispecific antigen binding polypeptide, or the genetically manipulated or engineered T cell, is formulated:
in a lipid formulation or a liposome or equivalents, in a liposomal vesicle, as a nanoparticle, or a nanoliposome, and optionally the lipid formulation or equivalent is injected, optionally is injected intramuscularly (IM), or into or approximate to a solid tumor,
in a lipid formulation as described in U.S. patent application no. US 20210046173 A1,
in a lipid formulation comprising or formulated as a liposome, or a lipid nanoparticle (LNP), or nanoliposome, that comprises: non-cationic lipids comprise a mixture of cholesterol and 1,2-distearoyl-sn-glycero-3-phosphocholine (DSPC), DSPC alone, 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG), or a polyethylene glycol (PEG)-lipid, or PEG-modified lipid, or an ionizable cationic lipid.

5-6. (canceled)

7: The method of claim 1, wherein the XCL1-expressing nucleic acid comprises the nucleic acid sequence (SEQ ID NO:1):
ATGAGGCTGCTGATCCTGGCCCTGCTGGGCATCTGCAGCCTGACCGCCT
ACATCGTGGAGGGCGTGGGCAGCGAGGTGAGCGACAAGAGGACCTGCGT
GAGCCTGACCACCCAGAGGCTGCCCGTGAGCAGGATCAAGACCTACACC
ATCACCGAGGGCAGCCTGAGGGCCGTGATCTTCATCACCAAGAGGGGCC
TGAAGGTGTGCGCCGACCCCCAGGCCACCTGGGTGAGGGACGTGGTGAG
GAGCATGGACAGGAAGAGCAACACCAGGAACAACATGATCCAGACCAAG
CCCACCGGCACCCAGCAGAGCACCAACACCGCCGTGACCCTGACCGGCT
AG.

8: The method of claim 1, wherein the XCL1-expressing nucleic acid comprises a nucleic acid having between about 90% to 99% sequence identity to SEQ ID NO:1 and encoding a polypeptide having XCL1 chemokine activity.

9: The method of claim 1, wherein the XCL1-expressing nucleic acid comprises a nucleic acid sequence that encodes the polypeptide:
(SEQ ID NO: 2)
MRLLILALLGICSLTAYIVEGVGSEVSDKRTCVSLTTQRLPVSRIKTYT
ITEGSLRAVIFITKRGLKVCADPQATWVRDVVRSMDRKSNTRNNMIQTK
PTGTQQSTNTAVTLTG.

10: The method of claim 1, wherein the XCL1-expressing nucleic acid comprises a nucleic acid sequence that encodes a polypeptide having between about 90% to 99% sequence identity to SEQ ID NO:2, or a polypeptide having about 92%, 95% or 97% sequence identity to SEQ ID NO:2, and having XCL1 chemokine activity.

11: The method of claim 1, wherein the T cell is a chimeric antigen receptor (CAR)-T cell.

12: The method of claim 11, wherein the CAR-T cell targets cancer cells.

13: The method of claim 2, wherein the expression vehicle or vector is selected from the group consisting of a herpes simplex virus, a human immunodeficiency virus (HIV), a synthetic vector, an adeno-associated virus (AAV), a lentivirus, an adenovirus and a plasmid.

14: The method of claim 13, wherein the AAV is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.
